SEQUENCE LISTING (14 sequences)

SEQ ID NO: 1 - DNA, 10 bp, Artificial Sequence
  Synthetic oligonucleotide
tcggtcagca

SEQ ID NO: 2 - DNA, 23 bp, Artificial Sequence
  Synthetic oligonucleotide
taacacttcatgggtcccgtagt

SEQ ID NO: 3 - DNA, 22 bp, Artificial Sequence
  Synthetic oligonucleotide
taacacttcatgggtcccgtag

SEQ ID NO: 4 - DNA, 22 bp, Artificial Sequence
  Synthetic oligonucleotide
catcatagtccagtgtccaggg

SEQ ID NO: 5 - RNA, 22 nt, Artificial Sequence
  Synthetic oligonucleotide
caucauaguccaguguccaggg

SEQ ID NO: 6 - DNA, 22 bp, Artificial Sequence
  Synthetic oligonucleotide
tcaagttcgcacttcctataca

SEQ ID NO: 7 - DNA, 22 bp, Artificial Sequence
  Synthetic oligonucleotide
tattttctgcattcgcccttgc

SEQ ID NO: 8 - DNA, 22 bp, Artificial Sequence
  Synthetic oligonucleotide
gacctgatgctgctggtgtgct

SEQ ID NO: 9 - DNA, 32 bp, Artificial Sequence
  Synthetic oligonucleotide
tgctgaccgaccctggacactggactatgatg

SEQ ID NO: 10 - DNA, 50 bp, Artificial Sequence
  Synthetic oligonucleotide
tgctgaccgaactacgggacccatgaagtgttaaaaaaaaaaaaaaaaaa

SEQ ID NO: 11 - DNA, 60 bp, Artificial Sequence
  Synthetic oligonucleotide
tgctgaccgaccctggacactggactatgatgaaaaaaaaaaaaaaaaaaaaaaaaaaaa

SEQ ID NO: 12 - DNA, 70 bp, Artificial Sequence
  Synthetic oligonucleotide
aaaaaaaaaaaaaaaaaaatgctgaccgatgtataggaagtgcgaacttgaaaaaaaaaaaaaaaaaaaa

SEQ ID NO: 13 - DNA, 80 bp, Artificial Sequence
  Synthetic oligonucleotide
aaaaaaaaaaaaaaaaaaaaaaaatgctgaccgagcaagggcgaatgcagaaaataaaaaaaaaaaaaaaaaaaaaaaaa

SEQ ID NO: 14 - DNA, 90 bp, Artificial Sequence
  Synthetic oligonucleotide
aaaaaaaaaaaaaaaaaaaaaaaaaaaaatgctgaccgaagcacaccagcagcatcaggtcaaaaaaaaaaaaaaaaaaaaaaaaaaaaa

(19) United States
(12) Patent Application Publication
(10) Pub. No.: US 2011/0159482 A1
(43) Pub. Date: Jun. 30, 2011

(54) METHOD FOR DETECTING MULTIPLE SMALL NUCLEIC ACIDS

(76) Inventors: Po-Ling CHANG, Xinzhuang City (TW); Yu-Sun CHANG, Linkou Shiang (TW); Shu-Jen CHEN, Taipei City (TW); Hua-Chien CHEN, Taipei City (TW)

(21) Appl. No.: 12/823,711

(22) Filed: Jun. 25, 2010

(30) Foreign Application Priority Data
Dec. 25, 2009 (TW) 98145130

(51) Int. Cl. C12Q 1/68

(52) U.S. Cl. 435/6

(57) ABSTRACT

The present invention discloses a method for simultaneously detecting multiple small nucleic acids, which comprises steps: mixing a specimen, fluorescent probes, and bridge nucleic acids having different lengths to form a tested liquid; hybridizing the mixed short nucleic acid molecules, probes and bridge nucleic acids; adding ligases to enable the ligations of the short nucleic acid molecules and the fluorescent probes with the bridge nucleic acids being the templates; injecting the tested liquid into a capillary, and applying a voltage to the capillary to generate an electrophoresis effect and separate the hybridization products; and using laser to induce different fluorescent rays from different reaction products, and measuring the fluorescent rays, whereby the present invention can simultaneously detect multiple types of short nucleic acid molecules in a single capillary.

### BACKGROUND OF THE INVENTION

1. Field of the Invention

The present invention relates to a method for analyzing small nucleic acids, particularly to a method for simultaneously detecting many types of small nucleic acids in a single capillary.

2. Description of the Related Art

A miRNA is a short-chain RNA (ribonucleic acid) consisting of about 22 nucleotides. The miRNAs are non-coding RNAs and have no direct correlation with transcription. However, the miRNAs play an important role in the post-transcriptional regulation. At present, researchers have found that miRNAs correlate with the differentiation, proliferation and canceration of cells. MiRNAs is also found to correlate with the intracellular regulation of the cells infected by viruses.

As miRNAs play more and more significant role in biological functions, how to detect miRNAs has become an important subject. The conventional Northern Blot method can detect miRNA more easily because the Northern Blot method is based on the gel electrophoresis technology and has a lower technological threshold a lower equipment threshold for biological researchers. However, the Northern Blot method is not necessarily an appropriate analysis method because of radiant ray, insufficient quantitative precision, and difficulty of automation. Further, how to standardize the quantitative data of different laboratories is also a big challenge for the laboratory personnel.

In recent years, the microarray chip has been the mainstream of miRNA analysis. The microarray chip has an advantage of high throughput. A microarray chip can detect more than one thousand miRNAs. In other words, a microarray chip can detect more than one thousand miRNAs. However, the professionals in the field still have some apprehension about the microarray.

RT-qPCR (Reverse Transcription-quantitative Polymerase Chain Reaction) is another method for detecting miRNAs. There has been a prior-art RT-qPCR-based miRNA detection system published in the periodical Nucleic Acids Res. However, the agents of an RT-qPCR test are very expensive. The expensiveness hinders RT-qPCR from simultaneously detect thousands of miRNAs of massive clinical specimens. The precision of PCR is due to the amplification effect of the polymerase reaction. However, the standard error increases with the amplification effect of the PCR reaction from the view point of the statistical analytical chemistry. A precision method for quantitatively analyzing miRNAs without using enzyme amplification is desired and deserves researching.

In the past two decades, the capillary electrophoresis has been extensively used to detect biological molecules, such as proteins, amino acids and DNAs (deoxyribonucleic acids). However, few of the capillary electrophoresis technologies are dedicated to miRNA analysis. Below are briefly described the capillary electrophoresis technologies for miRNA analysis. In 2003, Zhong, et al., proposed in the periodical Anal Chem. a technology of “Capillary Electrophoresis with Laser Induced Fluorescence (CE-LIF)”, which can directly evaluate the intracellular miRNA expression. In 2004, Tian, et al., proposed in the periodical Nucleic Acids Res. a technology able to simultaneously quantitatively analyze 44 genes. In 2004, Khan, et al., proposed, in the periodical Brain Res. Protoc., a technology which integrates RT-PCR and CE-LIF to quantitatively analyze miRNAs in the brain. In 2008, P.-L. Chang, et al., proposed in the periodical Anal Chem. a CE-LIF-based technology to detect the miRNAs of the Epstein-Barr virus in the nasopharyngeal carcinoma.

However, a very high-concentration polymeric buffer solution is required to directly separate the probe (22-nt) and the miRNAs with CE-LIF. Further, impurities are likely to appear in the synthesis and passivation processes of the fluorescent probe. In the conventional methods, the sample is thus very hard to accumulate in the case of insufficient resolution or the case of impurities existing. In 2007, Maroney, et al., proposed a splinted ligation-based technology to detect miRNAs. Similar to the Northern Blot method, the prior art also uses radioactive isotopes. Further, gel electrophoresis is not suitable for a quantitative or high-throughput test.

Accordingly, the present invention proposes a method for detecting multiple small nucleic acids, which can simultaneously detect multitudes of small nucleic acids in a single capillary with a single type of fluorescent probe.

### SUMMARY OF THE INVENTION

The primary objective of the present invention is to provide a method for detecting multiple small nucleic acids, which can simultaneously detect multitudes of small nucleic acids from the sample in a single capillary with a single type of nucleic acid probe, and which can perform a high-throughput test and greatly reduce the test cost.

Another objective of the present invention is to provide a method for detecting multiple small nucleic acids, which can recognize the specific features of individual bases precisely and complement the sequencing method.

A further objective of the present invention is to provide a method for detecting multiple small nucleic acids, which is exempt from enzyme amplification and has a simpler quality control process.

To achieve the abovementioned objectives, the present invention proposes a method for detecting multiple small nucleic acids, which comprises steps: providing a specimen containing a plurality of small nucleic acids; mixing the specimen, probes, and bridge nucleic acids having different lengths and complementary to the small nucleic acids and the probes; hybridizing the mixed nucleic acid molecules, probes and bridge nucleic acids in a splinted ligation method; adding ligases to enable the ligations of the nucleic acids and the probes; injecting the tested liquid containing the ligase into a capillary, and applying a voltage to the capillary to generate an electrophoresis effect and separate the products of the tested liquid; and using laser to induce different fluorescent rays from different reaction products, and measuring the fluorescent rays to detect the small nucleic acids in the specimen.

Below, the embodiments are described in detail in cooperation with the attached drawings to make easily understood the objectives, technical contents, characteristics and accomplishments of the present invention.

### BRIEF DESCRIPTION OF THE DRAWINGS

FIG. 1 is a flowchart of a method for detecting multiple small nucleic acids according to one embodiment of the present invention;

FIGS. 2(a)-2(c) are diagrams schematically showing a method for detecting multiple small nucleic acids according to one embodiment of the present invention;

FIGS. 3(a) and 3(b) are diagrams showing the fluorescent spectrums emitted in detecting a small nucleic acid BART7 according to one embodiment of the present invention;

FIGS. 4(a) and 4(b) are diagrams showing the fluorescent spectrums emitted in detecting multiple small nucleic acids according to one embodiment of the present invention;

FIG. 5 is a diagram showing the fluorescent spectrum emitted in detecting a small nucleic acid BART9-TcDNA according to one embodiment of the present invention; and

FIG. 6 is a diagram showing the fluorescent spectrum emitted in detecting a small nucleic acid BART9-cDNA according to one embodiment of the present invention.

### DETAILED DESCRIPTION OF THE INVENTION

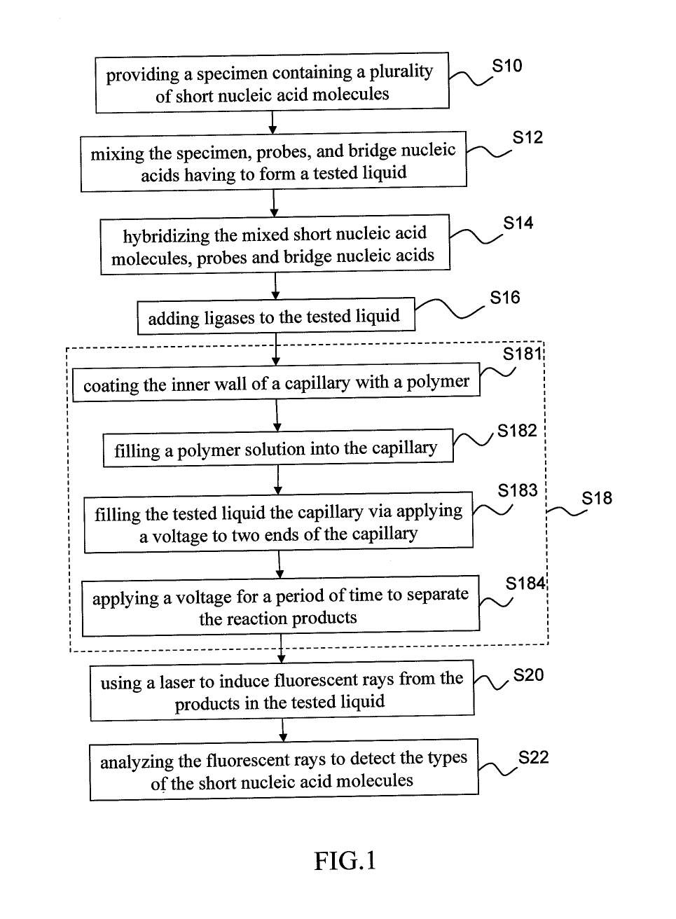

Refer to FIG. 1 for a flowchart of a method for detecting multiple small nucleic acids according to one embodiment of the present invention. In Step S10 is provided a specimen containing a plurality of unamplified short nucleic acid molecules, such as RNAs, DNAs, or the mixture of both. The abovementioned short nucleic acid molecules may be miRNAs, such as the genome of the Epstein-Barr virus. All the sequencing information used in the present invention is acquired from the 11^{th} edition database published by the Sanger institute.

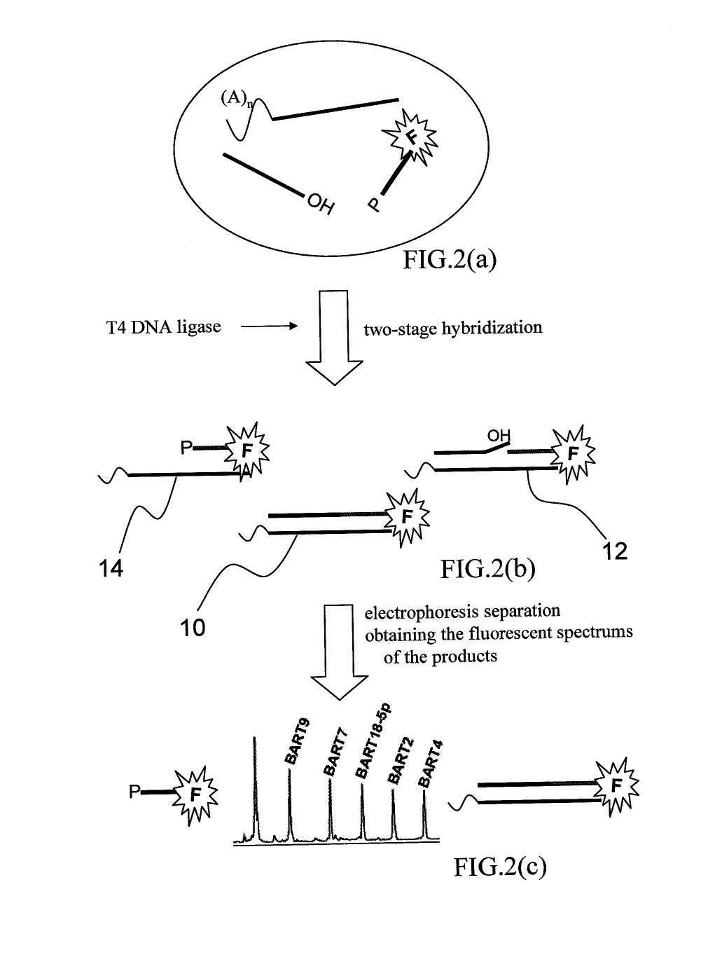

In Step S12 are mixed at least one probe, a plurality of bridge nucleic acids, and the specimen. Refer to FIG. 2(a). In one embodiment, the probe is 3′a fluorescence-labeled and 5′a phosphorylation polynucleotide. In other words, the probe is a single-strand nucleic acid with a synthesized fluorescence molecule (Alexa Fluor®532). The bridge nucleic acids are poly dA-tailed bridge DNAs. More exactly, the nucleic acids are poly deoxyadenosine polynucleotides. The probe, bridge nucleic acids and specimen are mixed to form a tested liquid. The sequences are completely complementary in the synthesized region between the short nucleic acids and the probe, and in the synthesized region between the short nucleic acids the bridge nucleic acids.

In Step S14 are hybridized the probe, the bridge nucleic acids and the short nucleic acids of the specimen in a splinted ligation reaction. The probe, miRNAs and bridge nucleic acids are dissolved in a magnesium ion-containing PCR buffer solution, and the tested liquid is agitated by gentle rotation. The tested liquid is heated to a theoretical fusion temperature to hybridize the short nucleic acid molecules of the specimen and the bridge nucleic acids. Then, the tested liquid is cooled to a temperature below the theoretical fusion temperature and maintained at the temperature to hybridize the probe and the bridge nucleic acids. The temperature cycle of heating the tested liquid is 70° C. for 15 minutes, 55° C. for 60 minutes, and 30° C. for 60 minutes.

In Step S16, a ligase and 1 μL of 10× ligase buffer solution are added into the tested liquid. In one embodiment, the ligase is a T4 DNA ligase. The ligase enables a ligation reaction at 16° C. for 30 minutes to connect the openings of the short nucleic acids and the probe. The products of complete ligation are washed with a centrifugal machine and a 70% ethanol solution at 4° C. Refer to FIG. 2(b). The products generated in Step 16 include a complete-ligation product 10, an incomplete-ligation product 12, and a hybridization product 14 of the residual probe and bridge nucleic acids. The complete-ligation product 10 is formed via ligating the openings of the short nucleic acids and the probe and then hybridizing the ligated short nucleic acids the probe with the bridge nucleic acids. In the incomplete-ligation product 12, the openings of the short nucleic acids and the probe are not ligated, and the unligated short nucleic acids and probe are respectively hybridized with the bridge nucleic acids. The surplus hybridization product 14 is formed via hybridizing the residual probe and bridge nucleic acids. All the products are dissolved in a Tris-Glycine buffer solution for the following steps (Step S18 and Step S20), which are based on a CE-LIF (Capillary Electrophoresis with Laser Induced Fluorescence) process.

In Step S18, the tested liquid processed by Step S16 is injected into a single capillary, and the reaction products are separated with electrophoresis. In Step S181, a 5% PVP aqueous solution is coated on the inner wall of the capillary before the specimen is injected. The capillary is a naked capillary made of fused quartz and having a diameter of 75 μm and a length of 50 cm (an effective length of 43 cm). In Step S182, a polymer solution is dissolved in a Tris-Glycine-Acetate buffer solution (2×TGA and pH7.0) containing 7M urea, and an injector fills the mixed solution into a capillary. In Step S183, the products of the tested liquid are filled into a single capillary with an electrokinetic injection method. Two ends of the capillary are inserted into a buffer solution containing a denaturant and a linear polymer. When electrophoresis occurs, the denaturant induces the hybridization of the probe and the bridge nucleic acids to denature without damaging the products of the ligation reaction. In Step S184, voltage is applied to the capillary to induce electrophoresis. A 200V/cm separating electric field is applied to separate the ligation reaction products filled into the anode via 10 kV electrokinetic injection. After the electric field has been applied for 10 seconds, the electrophoresis effect separates the products according to the lengths of the poly(dA) tails of the bridge nucleic acids.

Refer to FIG. 2(c). In Step S20, a laser is used to induce fluorescent rays from the products in the tested liquid, and the intensities of the fluorescent rays are continuously measured to obtain the relationships between the fluorescent intensities and the migration time. In one embodiment, a laser diode is powered by a high-voltage power supply to perform the experiment of inducing the fluorescent rays, wherein a 532 nm solid-state laser (Nd:YVO₄) is used to induce fluorescence from the products separated in the capillary. The experiments of electrophoresis and fluorescence induction are undertaken in a dark box. When Alexa Flour 32 is used as the fluorescence source, the scattered light is blocked by an OG550 intercept filter before the emitted rays reach the photoelectric cells. The amplified current is transmitted through a 10-kΩ resistor to a 10 Hz 24-bit A/D interface controlled by the software Clarity (DataApex, Prague, Czech Republic). The induced fluorescent rays are concentrated on a 20× object lens with an aperture of 0.25. The heterogeneous difference of the tested short nucleic acid molecules can be learned via analyzing the wavelengths and intensities of the fluorescent rays.

All the probes, small nucleic acids and bridge nucleic acids used in the present invention are the customized synthesized oligo-nucleic acids purchased from Integrated DNA Technologies, USA. The sequences of the oligo-nucleic acids are listed in Table.1.

TABLE 1

| Sequence | | | |
|---|---|---|---|
| ID | Name | Length | Sequence |
| 1 | Probe | 10 | TCGGTCAGCA |
| 2 | Short nucleic acid | 23 | TAACACTTCATGGGTCCCGTAGT |
| | molecule BART9 | | |
| 3 | Short nucleic acid | 22 | TAACACTTCATGGGTCCCGTAG |
| | molecule BART9-T | | |
| 4 | Short nucleic acid | 22 | CATCATAGTCCAGTGTCCAGGG |
| | molecule BART7 | | |
| 5 | Short nucleic acid | 22 | CAUCAUAGUCCAGUGUCCAGGG |
| | molecule BART7 RNA | | |
| 6 | Short nucleic acid | 22 | TCAAGTTCGCACTTCCTATACA |
| | molecule BART18_5p | | |
| 7 | Short nucleic acid | 22 | TATTTTCTGCATTCGCCCTTGC |
| | molecule BART2_5p | | |
| 8 | Short nucleic acid | 22 | GACCTGATGCTGCTGGTGTGCT |
| | molecule BART4 | | |
| 9 | Bridge nucleic acid | 32 | TGCTGACCGACCCTGGACACTGGACTATGATG |
| | Bridge-BART7 | | |
| 10 | Bridge nucleic acid | 50 | TGCTGACCGAACTACGGGACCCTGAAGTGTTA(17A) |
| | Bridge-BART9 + 17A | | |
| 11 | Bridge nucleic acid | 60 | TGCTGACCGACCCTGGACACTGGACTATGATG(28A) |
| | Bridge-BART7 + 28A | | |
| 12 | Bridge nucleic acid | 70 | (19A)TGCTGACCGATGTATAGGAAGTGCGAACTTGA(19A) |
| | Bridge-BART18_5p + 38A | | |
| 13 | Bridge nucleic acid | 80 | (24A)TGCTGACCGAGCAAGGGCGAATGCAGAAAATA(24A) |
| | Bridge-BART2_5p + 48A | | |
| 14 | Bridge nucleic acid | 90 | (29A)TGCTGACCGAAGCACACCAGCAGCATCAGGTC(29A) |
| | Bridge-BART4 + 58A | | |

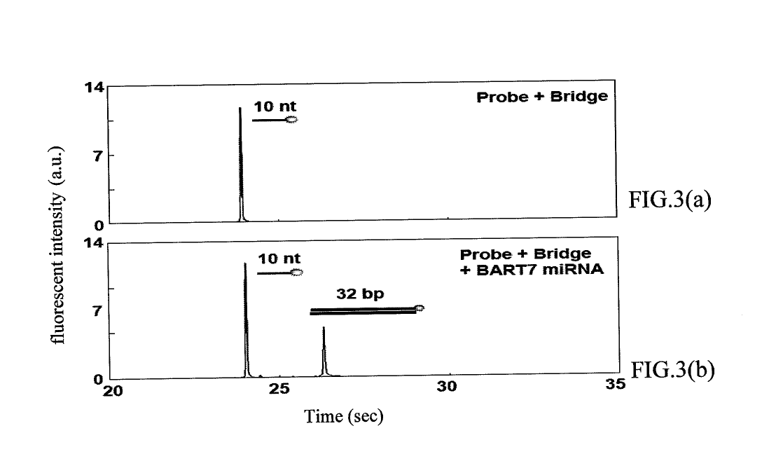

The present invention learns the information of the types of the short nucleic acid molecules in the specimen from the signals of the fluorescent rays. Refer to FIG. 3(a) and FIG. 3(b). The present invention detects a single small nucleic acid BART7 in the specimen. In FIG. 3(a), the peak is the signal of the fluorescent ray of the hybridization product of the probe and the bridge nucleic acid. In FIG. 3(b), the first peak is the signal of the fluorescent ray of the hybridization product of the probe and the bridge nucleic acid, and the second peak is the signal of the fluorescent ray of the ligated and hybridized probe, bridge nucleic acid and small nucleic acid BART7. Therefore, it is known that the specimen contains the small nucleic acid BART7.

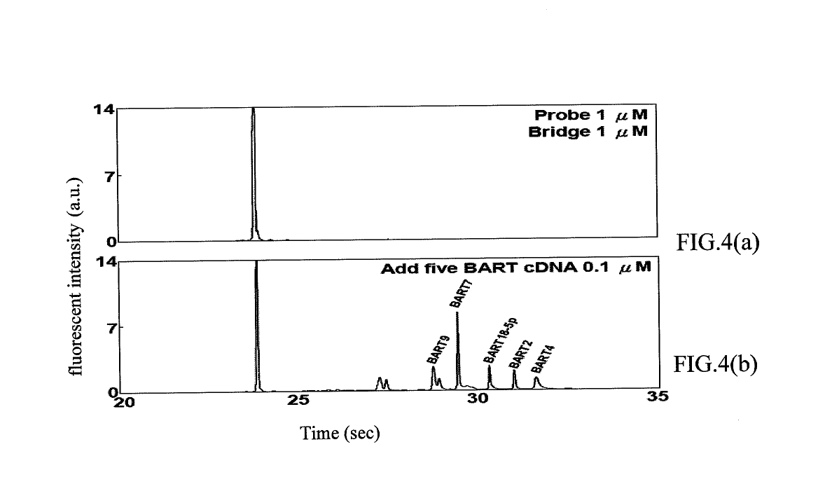

Refer to FIG. 4(a) and FIG. 4(b). The present invention can detect multiple types of small nucleic acids simultaneously. In FIG. 4(a), the peak is the signal of the fluorescent ray of the hybridization product of the probe and the bridge nucleic acid. In FIG. 4(b), the first peak is the signal of the fluorescent ray of the hybridization product of the probe and the bridge nucleic acid, and there are also the signals of the fluorescent rays of the ligation and hybridization products of the probe and bridge nucleic acid with the small nucleic acids: BART9, BART7, BART18-5P, BART2 and BART4. Therefore, it is known that the specimen contains five types of small nucleic acids: BART9, BART7, BART18-5P, BART2 and BART4.

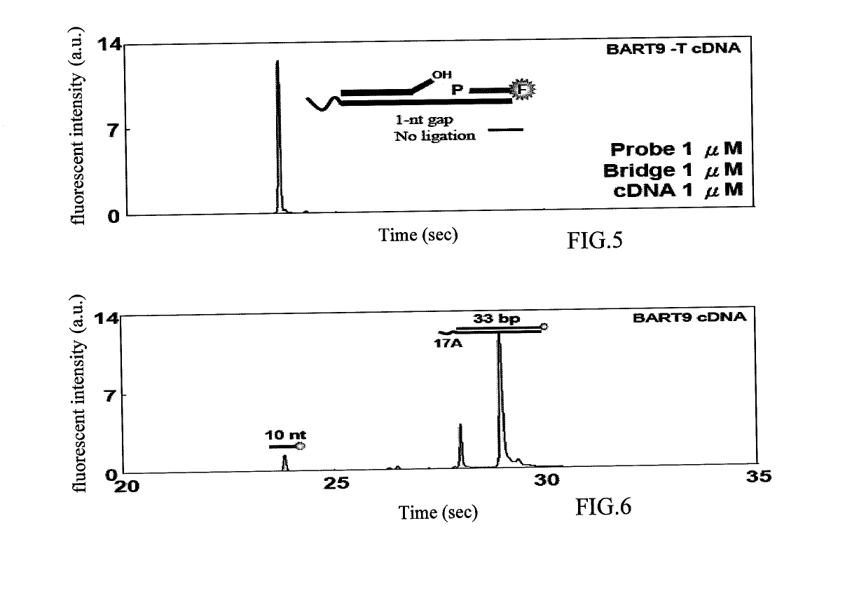

Refer to FIG. 5 and FIG. 6. The present invention can detect a short nucleic acid molecule and the (n−1)th nucleotide thereof. FIG. 5 shows that the specimen contains a short nucleic acid molecule BART9 cDNA. FIG. 6 shows that the specimen contains a short nucleic acid molecule BART9 cDNA, and that the sequence of BART9-T cDNA is the (n−1)th nucleotide of BART9 cDNA. Therefore, the present invention can discriminate BART9-T cDNA from BART9 cDNA.

In conclusion, the present invention proposes a method for detecting multiple small nucleic acids, wherein bridge nucleic acids with different lengths are hybridized with a probe and tested nucleic acids, and wherein a ligase is added to ligate the probe and the tested nucleic acids to form the ligation products, and wherein an electrophoresis technology and a laser-induced fluorescence technology are used to detect the tested nucleic acids in a capillary. Thereby, the present invention can simultaneously detect multiple types of small nucleic acids in a single capillary and achieve a high throughput with the experimental cost greatly reduced. Further, the present invention can recognize a single base of a small nucleic acid and can detect the lacking or increasing of the 3′a nucleotide. Thus, the present invention has the advantage of high recognizability. Furthermore, the present invention is exempted from enzyme amplification and has a simple quality control process. Therefore, the present invention has a high potential to be a mainstream method for detecting small nucleic acids.

The embodiments described above are only to demonstrate the technical contents and characteristics of the present invention to enable the persons skilled in the art to understand, make, and use the present invention. However, it is not intended to limit the scope of the present invention. Any equivalent modification or variation according to the spirit of the present invention is to be also included within the scope of the present invention.

1. A method for simultaneously detecting multiple small nucleic acids, comprising steps:
providing a specimen containing a plurality of unamplified short nucleic acid molecules;
mixing said specimen, a plurality of probes and a plurality of bridge nucleic acids to form a tested liquid, wherein each of said probes is a fluorescence-labeled polynucleotide, and wherein sequences of said bridge nucleic acids are completely complementary to ligated sequences of said probes and said short nucleic acid molecules;
performing a hybridization process on said probes and said bridge nucleic acids, and adding a plurality of ligases to enable ligation reactions to form a plurality of products;
separating said products; and
using a laser to induce fluorescent rays from said products, and measuring said fluorescent rays.

2. The method for simultaneously detecting multiple small nucleic acids according to claim 1, wherein said step of performing a hybridization process on said probes and said bridge nucleic acids, and adding a plurality of ligases to enable ligation reactions further comprises steps:
heating said tested liquid to denature and separate said tested liquid; and
cooling said tested liquid to cause recombination of said specimen, said probes and said bridge nucleic acids and complete said hybridization process.

3. The method for simultaneously detecting multiple small nucleic acids according to claim 2, wherein in said hybridization process, said tested liquid is heated to a theoretical fusion temperature to hybridize said short nucleic acid molecules and said bridge nucleic acids.

4. The method for simultaneously detecting multiple small nucleic acids according to claim 2, wherein in said hybridization process, said tested liquid is cooled to a temperature lower than a theoretical fusion temperature to hybridize said probes and said bridge nucleic acids.

5. The method for simultaneously detecting multiple small nucleic acids according to claim 2, wherein one of said ligases a T4 DNA ligase able to ligate openings of said short nucleic acid molecules to said probes.

6. The method for simultaneously detecting multiple small nucleic acids according to claim 5, wherein one of said short nucleic acid molecules and a (n−1)th nucleotide of said short nucleic acid molecule are recognized via said ligation reactions.

7. The method for simultaneously detecting multiple small nucleic acids according to claim 1, wherein said products include a complete-ligation product formed via ligating one said short nucleic acid molecule and an opening of one said probe and then hybridizing a ligation product of said short nucleic acid molecule and said probe with one said bridge nucleic acid.

8. The method for simultaneously detecting multiple small nucleic acids according to claim 1, wherein said step of separating said products further comprises steps:
injecting said products into a capillary placed in a buffer solution;
applying a voltage to said capillary to generate electrophoresis in said capillary;
maintaining said voltage for a predetermined interval of time; and
separating said products according to lengths of bases of said bridge nucleic acids.

9. The method for simultaneously detecting multiple small nucleic acids according to claim 8, wherein said buffer solution contains a denaturant.

10. The method for simultaneously detecting multiple small nucleic acids according to claim 9, wherein during said electrophoresis, said denaturant makes hybridization of said probes and said bridge nucleic acids denature without damaging products of said ligation reactions.

11. The method for simultaneously detecting multiple small nucleic acids according to claim 1, wherein said short nucleic acid molecules are selected from a plurality of micro ribonucleic acids.

12. The method for simultaneously detecting multiple small nucleic acids according to claim 11, wherein sequences of said micro ribonucleic acids are sequences of Epstein-Barr virus genomes.

13. The method for simultaneously detecting multiple small nucleic acids according to claim 1, wherein said probe is a deoxyribonucleic acid having a sequence identity of 1 and a length of 10; a sequence of said probe is TCGGTCAGCA (SEQ ID NO: 1).

14. The method for simultaneously detecting multiple small nucleic acids according to claim 1, wherein said short nucleic acid molecules are deoxyribonucleic acids respectively having sequence identities of from 2 to 8 and each having a length of 22 or 23, including BART9, BART9-T, BART7, BART7 RNA, BART18_{—}5p, BART2_{—}5p, and BART4.

15. The method for simultaneously detecting multiple small nucleic acids according to claim 1, wherein intensity of each fluorescent ray is continuously detected and presented as a function of migration time.

16. The method for simultaneously detecting multiple small nucleic acids according to claim 1, wherein said bridge nucleic acids are different types of poly dA-tailed bridge deoxyribonucleic acids.

17. The method for simultaneously detecting multiple small nucleic acids according to claim 16, wherein a length of each said bridge nucleic acid correlates with a length of each poly(dA) tail.

18. The method for simultaneously detecting multiple small nucleic acids according to claim 1, wherein said bridge nucleic acids are poly deoxyadenosine polynucleotides respectively having different lengths.

19. The method for simultaneously detecting multiple small nucleic acids according to claim 1, wherein said bridge nucleic acids are nucleic acid molecules respectively having sequence identities of from 9 to 14, including Bridge-BART7, Bridge-BART9+17A, Bridge-BART18_{—}5p+38A, Bridge-BART2_{—}5p+48A, Bridge-BART4+58A.
